(19) United States
(12) Patent Application Publication
(10) Pub. No.: US 2024/0105280 A1
(43) Pub. Date: Mar. 28, 2024

(54) COMPUTER-IMPLEMENTED METHOD AND APPARATUS FOR ANALYSING GENETIC DATA

(71) Applicant: GENOMICS PLC, Oxford (GB)

(72) Inventors: Rachel MOORE, Oxford (GB); Vincent Yann Marie PLAGNOL, Oxford (GB); Michael WEALE, Oxford (GB); Daniel WELLS, Oxford (GB); Christopher Charles Alan Spencer, Oxford (GB)

(73) Assignee: GENOMICS PLC, Oxford (GB)

(21) Appl. No.: 18/255,245

(22) PCT Filed: Nov. 26, 2021
(86) PCT No.: PCT/GB2021/053069

§ 371 Date: May 31, 2023

(30) Foreign Application Priority Data
Dec. 1, 2020 (GB) 2018905.6

(51) Int. Cl. G16B 20/20; G16B 40/20

(52) U.S. Cl. CPC G16B 20/20; G16B 40/20

(57) ABSTRACT

Disclosed is a method of analysing genetic data about an organism comprising receiving a plurality of input units. Each input unit comprises information about the association between genetic variants in a region of the genome and phenotypes or phenotype combinations. The method comprises carrying out iterations comprising, for each variant determining for which of the phenotypes or phenotype combinations the variant is causal based on the input units. If the variant is causal for phenotypes or phenotype combinations, a sampled effect size is determined of the variant on the phenotypes or phenotype combinations based on the input units and information about correlations between the variants in the region. For each variant, a prediction effect size is determined variant on the phenotypes or phenotype combinations based on an average across the iterations of the sampled effect sizes or of posterior effect sizes calculated using the sampled effect sizes.

The invention relates to analysing genetic and phenotype data about an organism to obtain information about the organism, particularly in the context of enabling improved polygenic risk scores (PRSs) to be obtained for phenotypes of interest.

A PRS is a quantitative summary of the contribution of an organism's inherited DNA to the phenotypes that it may exhibit. A PRS may include in its computation all DNA variants relevant (either directly or indirectly) to a phenotype of interest or may use its component parts if these are more relevant to a particular aspect of an organism's biology (including cells, tissues, or other biological units, mechanisms or processes). A PRS can be used directly, or as part of a plurality of measurements or records about the organism, to infer aspects of its past, current, and future biology.

PRSs are gaining traction as a tool for disease prevention, stratification and diagnosis. In the context of improving human health and healthcare, PRSs have a range of practical uses, which include, but are not limited to: predicting the risk of developing a disease or phenotype, predicting age of onset of a phenotype, predicting disease severity, predicting disease subtype, predicting the response to treatment, selecting appropriate screening strategies for an individual, selecting appropriate medication interventions, and setting prior probabilities for other prediction algorithms.

PRS may have direct use as a source of input in the application of artificial intelligence and machine learning approaches to making predictions or classifications from other high dimensional input data (for example imaging). They may be used to help train these algorithms, for example to identify predictive measurements based on non-genetic data. As well as having utility in making predictive statements about an individual, they can also be used to identify cohorts of individuals, included but not limited to the above applications, by calculating the PRS for a large number of individuals, and then grouping individuals on the basis of the PRSs.

PRSs can also aid in the selection of individuals for clinical trials, for example to optimise trial design by recruiting individuals more likely to develop the relevant disease or phenotypes, thereby enhancing the assessment of the efficacy of a new treatment. PRSs carry information about the individuals they are calculated for, but also for their relatives (who share a fraction of their inherited DNA). Information about the impact of an individual's DNA on their phenotypes can derive from any relevant assessment of the potential impact of carrying any particular combination of DNA variants.

In what follows we focus on the analysis of the recent wealth of information that derives from genetic association studies (GAS). These studies systematically assess the potential contribution of DNA variants to the genetic basis of a phenotype.

Since the mid-2000s, GAS (typically genome-wide association studies: GWAS, or association studies targeting single variants, or variants in a region of the genome, or GWAS restricted to a particular region of the genome) have been conducted on many thousands of (largely human) phenotypes, in millions of individuals, generating billions of potential links between genotypes and phenotypes. The resulting raw data is often then simplified to produce summary statistic data. GAS summary statistic data consists of, for each genetic variant (whether imputed or observed), the inferred effect size of the genetic variant on the phenotype of the GAS and the standard error of the inferred effect size. In other cases the individual level data, consisting of a full genetic profile of the individuals in a study and information about their phenotypes, may be available directly. However, individual level data is typically less widely available due to requirements on the privacy of an individual's data.

A PRS consists of the aggregation of the effects of a large number of genetic variants, typically each having small individual effects, to build an aggregate predictor for a trait of interest. PRSs can be calculated using effect sizes of variants determined from GWAS. Variants included in such a score can either be “causal variants”, in the sense that the variants directly affect a trait (weakly, but directly), or “tag variants”, which means that they are strongly correlated with other, unknown, variants that are causal, but that the tag variant itself does not have a direct effect on the phenotype.

Strategies for PRS construction are expanding, but a well-accepted general approach to building an accurate PRS consists of deconvoluting the signal in all regions of association by investigating the combination of variants that best capture the underlying biological associations. The number of associations will vary, with many genomic regions containing a single potential association while some genomic regions will contain multiple independent associations (up to 10 has been reported, though this is rare).

Some tools to build PRSs are designed to take advantage of summary statistics data. One approach, popularised by the LDpred software (Vilhjálmsson et al 2015, https://github.com/bvilhjal/ldpred), iterates through multiple random selections of plausible variants genome-wide based on a single GWAS and, as variants are picked or removed, estimates the residual signal.

Existing methods to deal with this issue are based upon creating PRS using training datasets from individuals exhibiting the trait (or phenotype) or combination of traits of interest. However, the amount of data that is available for particular phenotypes can vary greatly, both in quantity and quality. For example, where the trait of interest is chance of stroke, this can be difficult to quantify in a robust and consistent way. This affects in turn the usefulness of PRS calculated from studies of stroke risk. It would be advantageous to be able to analyse data from multiple studies in a way which improves the calculation of PRS for phenotypes of this kind.

It is an object of the invention to improve analysis of genetic data about an organism and/or allow more robust and/or accurate PRSs to be obtained for individuals.

According to an aspect of the invention, there is provided a computer-implemented method of analysing genetic data about an organism. The method comprises receiving a plurality of input units, wherein each input unit comprises information about the association between a plurality of genetic variants in a region of interest of the genome of the organism and one of a plurality of phenotypes or phenotype combinations of the organism, carrying out one or more iterations comprising, for each of the plurality of genetic variants: determining for which of the plurality of phenotypes or phenotype combinations the genetic variant is causal based on the plurality of input units; and if the genetic variant is determined to be causal for one or more of the phenotypes or phenotype combinations, determining a sampled effect size of the genetic variant on each of the one or more phenotypes or phenotype combinations based on the plurality of input units and information about correlations between the plurality of genetic variants in the region of interest, and for each genetic variant, determining a prediction effect size of the genetic variant on one or more of the phenotypes or phenotype combinations based on an average across at least a subset of the iterations of the sampled effect sizes of the genetic variant on the one or more phenotypes or phenotype combinations or of posterior effect sizes of the genetic variant for the input unit calculated using the sampled effect sizes.

By determining which variants are causal using data from a plurality of input units that relate to different phenotypes or phenotype combinations, the causal variants can be identified with greater confidence by including information from studies on related phenotypes or phenotype combinations. However, determining a prediction effect size separately for each input unit nonetheless allows the method to determine different effect sizes for different phenotypes or phenotype combinations. Thereby, the statistical power of using large datasets of high-quality data can be combined with the ability to generate phenotype-specific conclusions. By obtaining more accurate prediction effect sizes, more accurate PRS can consequently be calculated.

In some embodiments, determining for which of the plurality of phenotypes or phenotype combinations the genetic variant is causal comprises calculating a plurality of probabilities comprising: a probability of the information from the plurality of input units assuming that the genetic variant is not causal for any of the phenotypes or phenotype combinations; a probability of the information from the plurality of input units assuming that the genetic variant is causal for all of the phenotypes or phenotype combinations; and for one or more subsets of the phenotypes or phenotype combinations, a probability of the information from the plurality of input units assuming that the genetic variant is causal for the subset of phenotypes or phenotype combinations, and stochastically determining for which of the plurality of phenotypes or phenotype combinations the genetic variant is causal with a probability based on the plurality of probabilities. Using stochastic sampling allows the method to consider many different combinations of causal variants to identify an overall effect that best explains the observed data. Allowing variants to be causal for only a subset of the phenotypes or phenotype combinations can allow the method to account for phenotype-specific genetic mechanisms.

In some embodiments, the probability of the information from the plurality of input units assuming that the genetic variant is causal for one or more of the phenotypes or phenotype combinations is dependent on a proportion of the plurality of genetic variants expected to be causal, the plurality of input units, and a correlation between the effect sizes of the genetic variant on the phenotypes or phenotype combinations. In some embodiments, the probability of the information from the plurality of input units assuming that the genetic variant is not causal for any of the phenotypes or phenotype combinations is dependent on a proportion of the plurality of genetic variants expected to be causal, and the plurality of input units. In some embodiments, for each of the one or more subsets of the phenotypes or phenotype combinations, the probability of the information from the plurality of input units assuming that the genetic variant is causal for the subset of phenotypes or phenotype combinations is dependent on a proportion of the plurality of genetic variants expected to be causal, a subset of input units comprising the input units comprising information about the association between the plurality of genetic variants and one of the subset of phenotypes or phenotype combinations, and a correlation between the effect sizes of the genetic variant on the phenotypes or phenotype combinations. These terms allow pre-existing information about the proportion of variants that are causal to be incorporated in the analysis, and allow the prediction effect sizes between input units to vary. In the non-causal case, the effect sizes are zero, so no correlation between effects is appropriate.

In some embodiments, the proportion of the plurality of genetic variants expected to be causal is predetermined. In some embodiments, the correlation between the effect sizes of the genetic variant on the phenotypes or phenotype combinations is predetermined. Using predetermined values of the parameters allows pre-existing knowledge to be incorporated in the method in a computationally efficient manner.

In some embodiments, the proportion of the plurality of genetic variants expected to be causal is updated at each iteration. In some embodiments, the correlation between the effect sizes of the genetic variant on the phenotypes is updated at each iteration. Learning and updating the parameters at each iteration allows the method to converge on the true parameter values that may provide a more accurate result, but may be more computationally expensive.

In some embodiments, the input units are determined from respective groups of individuals, and each of the plurality of probabilities is dependent on one or more parameters quantifying an overlap in the groups of individuals between respective pairs of input units. Depending on the data used, some individuals may be present in multiple input units, which can distort the conclusions drawn. Adding parameters to account for this improves the accuracy of the resulting effect sizes.

In some embodiments, determining the sampled effect size of the genetic variant comprises calculating a probability distribution of effect sizes of the genetic variant on the one or more phenotypes or phenotype combinations, and sampling values of the effect sizes from the probability distribution. Using a probability distribution allows the method to sample different effect sizes, while still encouraging values to be chosen in a range considered most likely to be correct.

In some embodiments, the probability distribution is a multivariate normal distribution. Using a multivariate normal distribution provides a convenient way to allow different effect sizes for different input units. In some embodiments, the sampling of values of the effect size is performed using a Monte-Carlo Gibbs sampler. This type of sampling algorithm is particularly suited to the present application.

In some embodiments, the sampling of values of the effect size in each iteration is dependent on the sampled effect sizes from one or more previous iterations. This type of dependence can allow sampling to efficiently explore the space of possible values.

In some embodiments, the probability distribution is dependent on a correlation between the effect sizes of the genetic variant on the phenotype or phenotype combinations. This allows the likely range of differences in effect size between input units to be controlled to improve accuracy and computational efficiency.

In some embodiments, the correlation between the effect sizes of the genetic variant on the phenotypes or phenotype combinations is predetermined. Using predetermined values of the parameters allows pre-existing knowledge to be incorporated in the method in a computationally efficient manner.

In some embodiments, the correlation between the effect sizes of the genetic variant on the phenotypes or phenotype combinations is updated at each iteration. Learning and updating the parameters at each iteration allows the method to converge on the true parameter values which may provide a more accurate result, but may be more computationally expensive.

In some embodiments, determining the sampled effect sizes comprises using a model of causal relationships between the plurality of phenotypes or phenotype combinations. This allows pre-existing knowledge about directionality or magnitude of causal relationships between phenotypes to be incorporated in the analysis.

In some embodiments, each of the one or more iterations further comprises, for each genetic variant determined to be causal, subtracting weighted effect sizes from the information about the association between each other genetic variant and the phenotype or phenotype combination of each input unit; the weighted effect sizes being the sampled effect size of the genetic variant on the phenotype or phenotype combination of the input unit weighted by respective correlation factors between the genetic variant and each other genetic variant; and the correlation factors are determined based on the information about correlations between the plurality of genetic variants in the region of interest. Subtracting the effect of a variant determined to be causal from linked variants ensures that multiple causal variants are not erroneously identified based on a single causal relationship. Using input-unit specific correlation factors allows the method to account for variations in genetic correlations between subpopulations.

In some embodiments, carrying out one or more iterations comprises carrying out a predetermined number of iterations. Carrying out a predetermined number of iterations may provide adequate results for a known type of problem while remaining computationally efficient.

In some embodiments, each of the one or more iterations further comprises a step of evaluating a convergence parameter, and carrying out one or more iterations comprises carrying out iterations until a predetermined condition on the convergence parameter is met. Calculating a convergence parameter may be advantageous where an appropriate number of iterations is uncertain.

In some embodiments, the information about the association between the plurality of genetic variants and each of the phenotypes or phenotype combinations comprises, for each of the plurality of genetic variants, an estimate of a strength of association between the genetic variant and the phenotype or phenotype combination and an error in the estimate of the strength of association. As mentioned above, using this type of summary statistic data has advantages in the availability of large quantities of data.

According to another aspect, there is provided a method of determining a polygenic risk score for a target phenotype or target phenotype combination for a target individual. The method comprises: receiving genetic information about a region of interest of the genome of the target individual; receiving prediction effect sizes on the target phenotype or target phenotype combination of a plurality of genetic variants in the region of interest determined using the method of analysing genetic data of any preceding claim; and determining the polygenic risk score based on the genetic information for the target individual and the prediction effect sizes. As mentioned above, calculating polygenic risk scores is a particularly desirable use of the prediction effect sizes determined for genetic variants, and can be used for a variety of clinical applications.

According to another aspect of the invention, there is provided an apparatus for analysing genetic data about an organism. The apparatus comprises a receiving unit configured to receive a plurality of input units, wherein each input unit comprises information about the association between a plurality of genetic variants in a region of interest of the genome of the organism and one of a plurality of phenotypes or phenotype combinations of the organism, and a data processing unit configured to: carry out one or more iterations comprising, for each of the plurality of genetic variants: determining for which of the plurality of phenotypes or phenotype combinations the genetic variant is causal based on the plurality of input units; and if the genetic variant is determined to be causal for one or more of the phenotypes or phenotype combinations, determining a sampled effect size of the genetic variant on the one or more phenotypes or phenotype combinations based on the plurality of input units and information about correlations between the plurality of genetic variants in the region of interest; and for each genetic variant, determining a prediction effect size of the genetic variant on one or more of the phenotypes or phenotype combinations based on an average across at least a subset of the iterations of the sampled effect sizes of the genetic variant on the one or more phenotypes or phenotype combinations or of posterior effect sizes of the genetic variant for the input unit calculated using the sampled effect sizes.

The invention may also be embodied in a computer program comprising instructions which cause the computer to carry out the method, or a computer-readable medium comprising instructions which, when executed by a computer, cause the computer to carry out the method.

Embodiments of the invention will be further described by way of example only with reference to the accompanying drawings, in which:

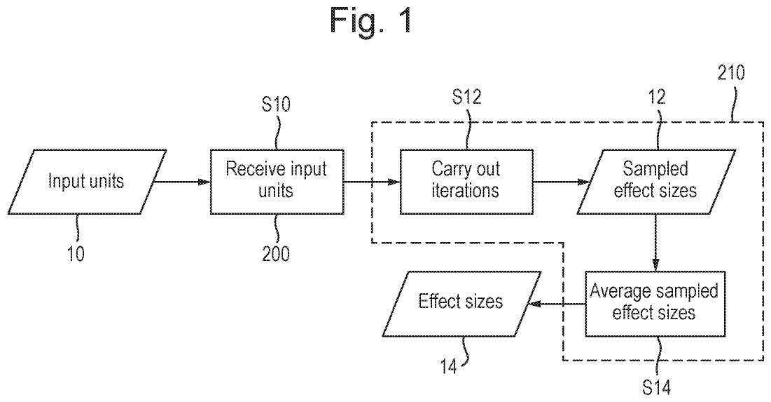

FIG. 1 is a flowchart of a method of analysing genetic data about an organism according to the invention;

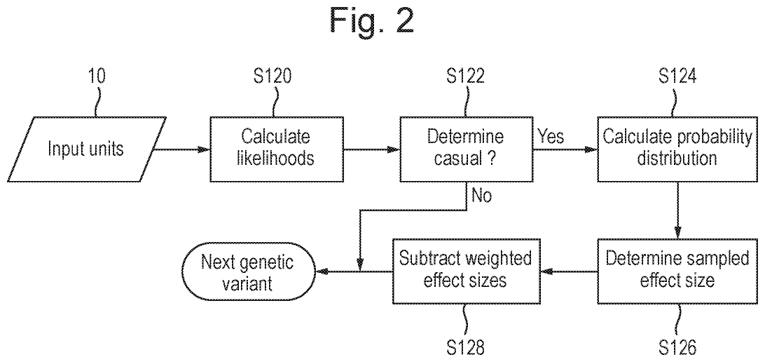

FIG. 2 is a flowchart showing the steps of each iteration in the step of carrying out iterations in the method of FIG. 1; and

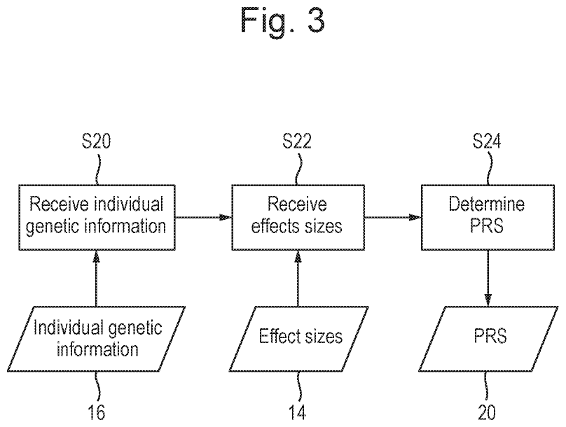

FIG. 3 is a flowchart of a method of determining a polygenic risk score according to the invention.

FIG. 1 shows a computer-implemented method of analysing genetic data about an organism. Typically, the organism is a human, although the method may be applied to other organisms. Although the method refers to “an organism” this may not refer to a specific individual organism, but to the organism or a group of organisms generically.

The method comprises a step S10 of receiving a plurality of input units 10. The input units 10 comprise information about the association between a plurality of genetic variants in a region of interest of the genome of the organism and a plurality of phenotypes or phenotype combinations of the organism. The plurality of phenotypes may include any physical, behavioural, or other phenotypes that may be of interest. The plurality of phenotype combinations may include combinations of any of the individual phenotypes. The genetic variants are typically single nucleotide polymorphisms, but may also comprise other types of genetic variation such as insertions or deletions of a section of the genome of the organism. In some embodiments, the plurality of phenotypes or phenotype combinations are phenotypes or combinations of phenotypes which are known or suspected to have a causal relationship with one another. Each of the input units will comprise information about the association between the plurality of genetic variants and one of the plurality of phenotypes or phenotype combinations.

Each input unit 10 may be derived from one or more genome-wide association studies (GWAS), and so may also be referred to as a study or a GWAS. Each input unit 10 will comprise information about the association between the plurality of genetic variants and the phenotype of the input unit 10 for a group of individuals, for example the individuals taking part in the corresponding GWAS.

In the embodiments described herein, the information about the association between the plurality of genetic variants and the phenotype or phenotype combination of the input unit 10 comprises, for each of the plurality of genetic variants, an estimate of a strength of association between the genetic variant and the phenotype or phenotype combination of the input unit 10 and an error in the estimate of the strength of association. Therefore, each input unit 10 comprises, for each variant i numbered 1 to n, an estimate \hat{β}_{ι} of the strength of association between the variant i and the phenotype or phenotype combination of the input unit, and a precision for that estimate, expressed as the standard error for the estimate SE [symbol] . This type of data is typically referred to as summary statistic data. A strength of summary statistics data is that the absence of limitation around sharing of individual level data due to privacy concerns means that much larger sample sizes can be made available for genetic analysis. However, in other embodiments, other types of information may be used, for example individual level data about all of the individuals in the groups from which the input units 10 are determined.

The estimates \hat{β}_{ι}, of the strength of association in each input unit 10 are marginal effect sizes estimated from each variant independently in the GWAS study. A key challenge is a consequence of correlations between genetic variants in the population. The marginal effect sizes may include contributions that are in fact due to other, correlated genetic variants within the region of interest. For example, if variant a and variant b appear together very often, and variant b increases the risk of the phenotype of the input unit 10 (i.e. is causal for that phenotype), an effect may also be attributed to variant a, because it appears often in individuals with the phenotype of the input unit 10. Hence a single causal variant will generate significant associations at many other variants, themselves not causal but only correlated to the causal variant.

It is desirable to determine the unknown true effect size βᵢ (or strength of association) at each given variant i, which is adjusted for correlations with nearby variants. The problem of genetic prediction consists of estimating that set of true effect sizes βᵢ. While all the \hat{β}_{ι} values are typically different from 0, the number of non-zero βᵢ values will typically be much smaller. The challenge facing many methods of analysing genetic data therefore consists of identifying the subset of K truly causal variants Xᵢ and their true strength of association βᵢ. The number of causal variants K is in general unknown. That collection of causal variants and their corresponding true effect sizes (Xᵢ, βᵢ) can be used to calculate the polygenic risk score for one or more of the plurality of phenotypes.

In the present method, the estimation of which variants are causal and their corresponding effect sizes is achieved by exploring the space of possible (Xᵢ, βᵢ) in the step S12 of carrying out one or more iterations. The details of this step will be discussed further below. In some embodiments, carrying out one or more iterations comprises carrying out a predetermined number of iterations. This may be advantageous if it is known approximately how many iterations are needed to obtain an accurate result. In some embodiments, each of the one or more iterations further comprises a step of evaluating a convergence parameter, and carrying out one or more iterations comprises carrying out iterations until a predetermined condition on the convergence parameter is met. This may be advantageous if it is uncertain how many iterations will be required to give an accurate result.

As mentioned above, currently available methodologies for analysing genetic data (such as LDpred) consider one GWAS at a time and perform random sampling of which variants are causal for a target phenotype, for example by Monte Carlo sampling. LDpred relies on being able to solve a Bayesian computation for one study and one genetic variant. It then uses a Gibbs sampling technique to extend the methodology from one to multiple correlated variants. Precisely, for a given genetic variant, LDpred uses a prior assumption that:
with probability (1-p) the effect of the genetic variant on the target phenotype is 0 (i.e. the variant is not causal).
with probability p the effect on the target phenotype is normally distributed with mean 0 and variance σ² (i.e. the variant is causal with a distribution of effect sizes centred around 0).

With these assumptions, and the summary statistics \hat{β}_{ι}, SE [symbol] in a training GWAS for the target phenotype, it is possible to derive an analytical formula for the posterior distribution of the true effect size βᵢ on the target phenotype, and to sample from this distribution to estimate the true effect size.

However, this approach has limitations particularly for smaller studies that can lead to poor or suboptimal results for some phenotypes or phenotype combinations. Studies on some phenotypes or phenotype combinations may be small or of low quality due to the difficulty of assessing the phenotype in a consistent and quantitative manner, leading to poor predictive results for those phenotypes. For example, when studying the genetics of heart attack (coronary artery disease, CAD), collecting cohorts of heart attack patients is challenging. It is more straightforward to measure blood lipids, which can be done in large cohorts systematically. It is established that a genetic variant that increases levels of a subtype of blood lipids, called low density lipoprotein (LDL), is very likely to contribute to heart attack risk. It is therefore beneficial to jointly analyse studies that describe the genetics of blood lipids together with the genetics of heart attack, in order to derive valuable information from the associations between the two phenotypes. This is not something that can be done if only a single study is analysed at a given time, as in most existing methods.

When considering multiple studies, currently available methods consist of combining the multiple studies into a single meta-analysis, and performing further processing, for example determining a PRS, on that meta-analysis. An example of a tool that accounts for evidence of association between variants and a target phenotype based on multiple studies is multi-trait analysis of GWAS (MTAG, Turley et al 2018). MTAG combines a set of GWAS and generates, for each input GWAS, a type of meta-analysis that results in updated summary statistics per input GWAS. These updated summary statistics can be fed into any standard PRS construction methodology, including LDPred (Craig et al, Nature Genetics 2020). However, MTAG makes fixed global assumptions for all of the variants in the genome, including the prior assumption on the phenotypic variance a variant explains and the degree of correlation between the effect sizes of two studies. These assumptions are often incorrect. In the example of using LDL and CAD to predict CAD, there are some variants that are causal for both CAD and LDL and other variants that are causal only for CAD, which violates the constant correlation assumption used in MTAG. In addition MTAG uses the marginal summary statistics without simultaneously accounting for LD information, meaning that the method is not fully leveraging the richness of the input datasets.

Another existing approach to combining multiple studies is the single variant Bayesian computation developed in another context (Trochet et al, Genetic Epidemiology 2019). In this method, the aim is not prediction of effect sizes, but the combining of studies to increase power to detect genetic associations. Hence, genetic variants are considered individually and there is no motivation to control for the correlation pattern between them.

To overcome these limitations, the present method allows information from multiple studies on multiple phenotypes or phenotype combinations to be combined when determining causal variants and their effect sizes, but, significantly, allows the determined effect sizes of each genetic variant to differ between input units 10. This allows the greater statistical power of larger, more robust studies to be used together with the data from other studies on a phenotype or phenotype combination of interest to improve estimations of which variants are causal for the phenotype or phenotype combination of interest, but nonetheless derive effect sizes specific to the phenotype or phenotype combination of interest.

This involves extending the Bayesian computation from LDPred (Vilhjálmsson et al 2015) from one study to an arbitrary number of studies for multiple different phenotypes. In doing so, a link is made between the single variant multi-studies work of Trochet et al and the multi-variants single study work of Vilhjálmsson et al. By understanding the relationship between both methodological approaches, it becomes possible to integrate in a flexible manner multiple studies and to create a prediction algorithm based on multiple GWAS, rather than a single study.

As shown in FIG. 2, each iteration in the step S12 of the present method comprises, for each of the plurality of genetic variants, determining for which of the plurality of phenotypes or phenotype combinations the genetic variant is causal based on the plurality of input units 10. As for existing methods, genetic variants are considered one by one, for example in physical order or by random sampling, though other options are possible. However, at each variant, the present method incorporates multiple studies rather than a single study, and assesses the probability of models of the causality and effect size of the variant on each of the input units 10 (for example by Bayesian analysis, as discussed further below). Therefore, the present method determines for which phenotypes or phenotype combinations each genetic variant is causal by analysing all of the input units 10 together, not by considering only one input unit 10 at a time, or by combining the input units 10 into a single meta-analysis as in existing methods.

An important distinction relative to some of the existing methods described above is that in the present method, the method allows for some but not all causal variants to be shared across input units 10. This allows for the method to model effectively the complexity of cross-phenotype causal relationships.

If the genetic variant is determined to be causal for one or more of the phenotypes or phenotype combinations, a step is performed of determining a sampled effect size 12 of the genetic variant on the one or more phenotypes or phenotype combinations for each of the input units 10 based on the plurality of input units 10 and information about correlations between the plurality of genetic variants in the region of interest. Therefore, in the exploration of the space of causal variants and joint effect sizes, when a variant is selected as causal for one or more phenotypes or phenotype combinations, different effect sizes are sampled for each phenotype.

In the embodiment of FIG. 1, determining for which of the plurality of phenotypes or phenotype combinations the genetic variant is causal comprises a step S120 of calculating a plurality of probabilities, and a step S122 of stochastically determining for which of the plurality of phenotypes or phenotype combinations the genetic variant is causal with a probability based on the plurality of probabilities. The plurality of probabilities comprises a probability of the information from the plurality of input units assuming that the genetic variant is not being for any of the phenotypes or phenotype combinations, a probability of the information from the plurality of input units assuming that the genetic variant is causal for all of the phenotypes or phenotype combinations, and for one or more subsets of the phenotypes or phenotype combinations, a probability of the information from the plurality of input units assuming that the genetic variant is causal for the subset of phenotypes or phenotype combinations.

In step S120, the probability of the information from the plurality of input units assuming that the genetic variant is causal for all of the phenotypes or phenotype combinations may be dependent on a proportion of the plurality of genetic variants expected to be causal, the plurality of input units 10, and a correlation between the effect sizes of the genetic variant on the phenotypes or phenotype combinations for each of the input units 10. The probability of the information from the plurality of input units assuming that the genetic variant is not causal for any of the phenotypes or phenotype combinations may be dependent on a proportion of the plurality of genetic variants expected to be causal, and the plurality of input units 10. For each of the one or more subsets of the phenotypes or phenotype combinations, the probability of the information from the plurality of input units assuming that the genetic variant is causal for the subset of phenotypes or phenotype combinations may be dependent on a proportion of the plurality of genetic variants expected to be causal, a subset of input units 10 comprising the input units 10 comprising information about the association between the plurality of genetic variants and one of the subset of phenotypes or phenotype combinations, and a correlation between the effect sizes of the genetic variant on the phenotypes. The probabilities may be combined with prior values.

For example, consider a situation where a PRS for stroke is desired, and two input units 10 are available comprising information about, respectively, the association between the plurality of genetic variants and blood pressure, and the association between the plurality of genetic variants and stroke risk. The present method can model the fact that a variant that increases blood pressure will always increase the risk of stroke, but the converse is not necessarily true.

In the stroke example, three alternative configurations may be considered for any given variant:
a null hypothesis that, with probability p₀=(1-p₁-p₂), the variant has a 0 effect size for the phenotypes of all of the input units 10;
a first alternative that, with probability p₁, the effect sizes of the genetic variant for the phenotypes of the two input units 10 follow a multivariate Gaussian distribution, i.e. that the genetic variant is causal for both stroke and blood pressure; and
a second alternative that, with probability p₂, the effect size of the genetic variant on the stroke input unit 10 follows a Gaussian distribution, and the effect size of the genetic variant on the blood pressure input unit 10 is 0, i.e. that the genetic variant is causal for stroke only.

These priors can then be combined with the probabilities mentioned above for each case dependent on the other relevant factors.

As well as single phenotypes, such as stroke risk and blood pressure as in the example above, input units may relate to a combination of two or more phenotypes. In this case, each input units comprises information about the association between a plurality of genetic variants in a region of interest of the genome of the organism and one of a plurality of phenotype combinations of the organism. For example, input units 10 may relate to a combination of blood pressure and gender, so that separate input units 10 are used for blood pressure in males and blood pressure in females. The method may then choose between different alternative configurations of causality of a variant for the particular combinations of phenotypes. For example, some variants may be causal for high blood pressure in both males and females, while other variants may be causal for high blood pressure in males but not in females. The present method allows the information about causality in the different groups to be leveraged jointly to improve estimates of effect size for both groups.

Another example is that variants contributing to addiction are associated with lung cancer, because they mediate smoking. But if an individual is not a smoker, one would want to consider a PRS that does not include addiction related genetic information. Therefore, the method may consider two different phenotype combinations: lung cancer in smokers and lung cancer in non-smokers (i.e. a combination of the lung cancer phenotype with the behavioural phenotype of smoker/non-smoker). Then three probabilities are calculated for each genetic variant: a probability of the information from the plurality of input units assuming the genetic variant not being causal (i.e. not relevant for any type of lung cancer), a probability of the information from the plurality of input units assuming the genetic variant is causal for all of the input units (i.e. “shared” between smoker and non-smoker lung cancer), and a probability of the information from the plurality of input units assuming the genetic variant is causal for a subset of only the input unit 10 from smokers (i.e. the variant is causal for smoker lung cancer only). In this situation the two categories (“lung cancer in smokers” and “lung cancer in non-smokers”) are two different phenotype combinations. Therefore the method may determine different sets of causal variants for their corresponding input units 10. This allows the statistical power of the larger smoker-inclusive studies to be used to improve estimates of the causal variants, while still allowing for the fact that some variants (such as the addiction relation variants) may not be causal for non-smokers.

The parameter p_{c} is the proportion of the plurality of genetic variants expected to be causal under a given configuration. In some embodiments, the proportion of the plurality of genetic variants expected to be causal is predetermined. This may be more computationally efficient if an estimate is available. Alternatively, a grid of values of p_{c} can be considered and the optimum parameter value for p_{c} can be selected by maximising prediction in a dataset of individual level data with outcomes. In some embodiments, the proportion of the plurality of genetic variants expected to be causal is updated at each iteration. This allows the method to converge on the true value of p_{c} which potentially improves accuracy.

Under the null hypothesis, the value of the sampled effect size 12 is equal to 0 for all input units 10. Therefore, the covariance matrix for the sampled effect sizes βᵢ of the variant is only driven by the uncertainty in the value of the parameters (referred to as SE_{i,j}, for the standard error of the marginal effect size of variant i from input unit j), which is itself a function of the sample size of the study and encoded in the summary statistics of the input unit 10. Precisely we have:

$$\\ \begin{matrix}{V}_{i} = [ \begin{matrix}{SE}_{i , 1}^{2} & 0 & 0 & 0 \\ 0 & {SE}_{i , 2}^{2} & 0 & 0 \\ 0 & 0 & … & 0 \\ 0 & 0 & 0 & {SE}_{i , m}^{2}\end{matrix} ] & ( 1 )\end{matrix}$$

where SE_{i,j} refers to the standard error for the variant i and input unit j, where there are m input units 10 in total.

Under the alternatives, the sampled effect size βᵢ of the variant i is non-zero, and distributed as a multivariate Gaussian (with a dimensionality appropriate to the number of phenotypes for which the variant is determined to be causal, i.e. the number of phenotypes in the subset) with mean 0 and a plurality of unknown variances σⱼ² for each dimension of the multivariate Gaussian. In each of the alternative configurations c, there is a new specification:

[symbol]

=Σ_{i,c}+Vᵢ (2)

where

$$\\ \begin{matrix}{\sum }_{i , c} = [ \begin{matrix}{σ}_{1}^{2} & {ρ}_{i} {σ}_{1} {σ}_{2} & ⋯ & {ρ}_{i} {σ}_{1} {σ}_{m} \\ {ρ}_{i} {σ}_{1} {σ}_{2} & {σ}_{2}^{2} & ⋯ & {ρ}_{i} {σ}_{2} {σ}_{m} \\ ⋯ & ⋯ & ⋯ & ⋯ \\ {ρ}_{i} {σ}_{1} {σ}_{m} & {ρ}_{i} {σ}_{1} {σ}_{m} & ⋯ & {σ}_{m}^{2}\end{matrix} ] & ( 3 )\end{matrix}$$

with ρᵢ being the correlation between the effect sizes of the genetic variant i on the target phenotype for each of the m input units 10. In each alternative configuration c, the variance σⱼ² will be zero for any input unit j for which the variant i is not causal under that configuration. In some embodiments, the correlation ρᵢ between the effect sizes of the genetic variant on the target phenotype or phenotype combination for each of the input units 10 is predetermined, which may be more computationally efficient. The predetermined value may be based on existing, external data if it allows an apriori estimation of how strongly the effects for different phenotypes or phenotype combinations should be correlated.

In other embodiments, the correlation between the effect sizes of the genetic variant on the phenotype or phenotype combination of each of the input units 10 is updated at each iteration. This allows the method to converge on the true correlation coefficients, potentially leading to more accurate results. Alternatively, a grid of values of the correlation can be considered and the optimum parameter value for these correlations can be selected by maximising prediction in a dataset of individual level data with outcomes. In the example given here, the correlation between the effect sizes is a single parameter that is the same for all combinations of input units 10.

The correlation may also be a correlation matrix, allowing for the correlations to differ between different combinations of input units 10. This can be used to account for different expectations of the strength (or presence) of the causal relationships between particular phenotypes or phenotype combinations.

In an embodiment of step S122, for each variant i the posterior odds Odds_{i,k} of belonging to a particular configuration k from the C possible configurations can be calculated using the probabilities determined in step S120:

$$\\ \begin{matrix}{Odds}_{i , k} = \frac{f ( {β}_{i} , {V}_{i} + {\sum }_{i , k} ) \times {p}_{k}}{f ( {β}_{i} , {V}_{i} ) \times {p}_{0} + {\sum }_{c = 1 , c \neq k}^{C} f ( {β}_{i} , {V}_{i} + {\sum }_{i , c} ) \times {p}_{c}} & ( 4 )\end{matrix}$$

The odds used to stochastically determine which configuration the variant belongs to (i.e. for which of the plurality of phenotypes the genetic variant is causal) are then computed as shown in equation (4). βᵢ in these equations is a vector of dimension m, i.e. it specifies an effect of variant i on each of the m input units 10.

Where the input units 10 are determined from respective groups of individuals, and depending on the studies that are used to determine the input units 10, one potential issue is sample overlap across studies. For example, a stroke risk study may be used to derive one input unit 10, and is consequently analysed jointly with input units 10 derived from another blood pressure study. Some of the individuals in the group of individuals used to perform the stroke risk study may also be present in the group of individuals of the blood pressure study. For example, the group of individuals of the stroke risk study may be a subset of the blood pressure study set. To account for this, in some embodiments, each of the plurality of probabilities is dependent on one or more parameters quantifying an overlap in the groups of individuals between respective pairs of input units 10.

For example, one way to account for that possibility is to update the covariance matrix Vᵢ shown above to become:

$$\\ \begin{matrix}{V}_{i} = [ \begin{matrix}{SE}_{i , 1}^{2} & {r}_{1 , 2} {SE}_{i , 1} {SE}_{i , 2} & ⋯ & {r}_{1 , m} {SE}_{i , 1} {Se}_{i , m} \\ {r}_{1 , 2} {SE}_{i , 1} {SE}_{i , 2} & {SE}_{i , 2}^{2} & ⋯ & {r}_{2 , m} {SE}_{i , 1} {SE}_{i , m} \\ ⋯ & ⋯ & ⋯ & ⋯ \\ {r}_{1 , m} {SE}_{i , 1} {SE}_{i , m} & {r}_{2 , m} {SE}_{i , 1} {SE}_{i , m} & ⋯ & {SE}_{i , m}^{2}\end{matrix} ] & ( 5 )\end{matrix}$$

where the r_{x,y} coefficients account for the overlap in samples across studies, and (as will be discussed further below) also model correlations across the sampled effect sizes 12 due to sharing of samples. To clarify notations, these r_{x,y} have no relationship to the correlation factors r_{i,j} describing variant level correlation (which will be discussed in more detail below). This addition (described in Trochet et al 2019) is important in practice to achieve accurate results, although it is not essential and adequate results may still be achieved without it.

If the genetic variant is determined to be causal for one or more of the phenotypes or phenotype combinations, a posterior mean and variance can be computed for the joint effect size across all of the one or more phenotypes or phenotype combinations. The step of determining a sampled effect size 12 of the genetic variant comprises a step S124 of calculating a probability distribution of effect sizes of the genetic variant on the one or more phenotypes or phenotype combinations, and a step S126 of sampling values of the effect sizes from the probability distribution.

A sampled effect size 12 is used because in practice it is impossible to fully explore the space of all possible causal variants and all possible corresponding effect sizes in a reasonable time. Therefore, sampling techniques, for example Monte Carlo simulations, are used to explore the space of causal variants and their corresponding effect sizes. In some embodiments, the sampling of values of the effect size in each iteration is dependent on the sampled effect sizes 12 from one or more previous iterations. This can be used to guide the sampling technique to adequately explore the space of possible values. In some embodiments, the sampling of values of the effect size is performed using a Monte-Carlo Gibbs sampler.

Determining the sampled effect sizes may comprise using a model of causal relationships between the plurality of phenotypes or phenotype combinations. This can be introduced using the correlations between the effect sizes of the phenotypes, for example using a matrix of correlations as mentioned above. This causal relationship can also be used when determining the plurality of probabilities.

In a preferred embodiment, the probability distribution is a multivariate normal distribution. The probability distribution may be dependent on a correlation between the effect sizes of the genetic variant on the phenotypes or phenotype combinations of each of the input units 10. As discussed for the probabilities above, the correlation between the effect sizes of the genetic variant on the phenotypes of each of the input units 10 may be predetermined. Alternatively, the correlation between the effect sizes of the genetic variant on the phenotypes may be updated at each iteration, allowing the method to converge on the true value of the correlation.

In a specific example where the variant is determined to belong to configuration k, the probability distribution is the posterior mean for the effect size, and is distributed as a multivariate normal distribution:

βᵢ˜MVN((Σ_{i,k}⁻¹+Vᵢ⁻¹)⁻¹(Vᵢ⁻¹βᵢ),(Σ_{i,k}⁻¹+Vᵢ⁻¹)⁻¹) (6)

A technical challenge in identifying the correct combination of causal variants in a region of the genome is that variants can be correlated with each other. Therefore, an important step in some embodiments of methods for analysing genetic data with the aim of calculating a PRS is the ability to control for correlations between genetic variants. As mentioned above, correlations between variants can cause some variants to have large marginal effect sizes in the input unit 10 even when they are not causal for the phenotype or phenotype combination of the input unit 10.

To account for this, in some embodiments, each of the one or more iterations further comprises, for each genetic variant determined to be causal, a step S128 of subtracting weighted effect sizes from the information about the association between each other genetic variant and the phenotype or phenotype combination of each input unit 10. Hence when a genetic variant i is determined to be causal, and a sampled effect size βᵢ is determined for the genetic variant i, the effect of that causal variant is subtracted from surrounding correlated variants. The weighted effect sizes are the sampled effect size 12 of the genetic variant on the phenotype or phenotype combination of the input unit 10 weighted by respective correlation factors between the genetic variant and each other genetic variant j.

In a particular embodiment, this results in the following correction being applied to the marginal effect sizes of each of the other genetic variants j:

$$\\ \begin{matrix}{β}_{j}^{corrected} = {\hat{β}}_{j} - {\sum }_{i} {r}_{i , j} {β}_{i} & ( 7 )\end{matrix}$$

In the formula above, the βᵢ are the sampled effect sizes 12 of each of the variants currently determined to be causal. The values r_{i,j} are correlation factors that describe the correlation between each pair of variants i and j. The correlation factors are determined based on the information about correlations between the plurality of genetic variants in the region of interest, which may be estimated from a reference set of reference sequences. This correction formula assumes that each genotyped variant Xᵢ has been normalized to have variance 1, and that its associated marginal effect size \hat{β}_{ι} has been updated accordingly. If this is not the case, an additional correction needs to be applied to account for the standard error for each estimated effect size.

The effect of this correction is that, when it is determined whether a variant is causal, its marginal effect size will be corrected using the formula above based on the sampled effect sizes of all the variants so far determined to be causal in that iteration. Therefore, in such embodiments, the effect size βᵢ used in equations (4) and (6) will actually be the corrected effect size calculated using equation (7). A significant subtlety is that this subtraction step for a particular genetic variant depends on which other variants have been sampled as causal at the point the subtraction is performed. Therefore, some variation in βᵢ can arise between iterations depending on the order in which genetic variants are sampled.

Importantly, it is often not possible to calculate the correlation factors between genetic variants (the values r_{i,j} in the example above) directly from the data itself and instead must originate from a reference population, such as data generated by the 1,000 Genomes consortium. The set of these correlation factors may be referred to as a linkage disequilibrium map (or LD map), and reflects a covariance structure between the genetic variants. These correlation factors may vary between subpopulations. For example, individuals of European ancestry may have different patterns of LD to individuals of South-East Asian ancestry. Therefore, inferences made for one subpopulation, or made based on data from individuals from a mixture of subpopulations, are unlikely to be as precise for different subpopulations. For example, the datasets that support the construction of PRSs are often based on large cohorts of European ancestries. As a result, these scores often perform poorly in non-European ancestries. In existing methods, which only analyse a single study those correlation factors will be determined from a reference population LD map matching the population of origin for the study.

In the present method, the effect size subtraction step S128 may be performed in a way that accounts for correlations across genetic variants in a manner that is consistent with ancestry-specific patterns of variant correlations. The present method may, where appropriate, handle in parallel multiple reference LD maps. Once a variant is determined to be causal for one or more phenotypes, the subtraction step S128 is then applied in an ancestry-specific manner. Therefore, where the input units 10 are determined from respective groups of individuals, the correlation factors between the genetic variant and each other genetic variant depend on an ancestry of the group of individuals of the input unit 10. A one-to-one mapping may be used between the ancestry where each study was performed and its matching LD map (covariance structure).

For example, where the group of individuals of at least one of the input units 10 comprises individuals having a common ancestry, the correlation factors are determined based on correlations between genetic variants in the region of interest for individuals having the common ancestry.

In another example, the plurality of input units 10 are derived from studies that contain individuals from a mixture of ancestries. Where the group of individuals of at least one of the input units 10 comprises individuals having different ancestries, the correlation factors are determined based on an average of correlations between genetic variants in the region of interest for individuals having each of the different ancestries. The method determines the LD map for the mixed input units 10 to be an average of plural “primary” LD maps, each of these “primary” LD maps being determined from a well-defined reference ancestry set of correlations between genetic variants.

It is possible that, depending on the input data used, not all of the plurality of genetic variants may exist at meaningful frequencies for all ancestries. For example, some genetic variants may only be found in individuals of a specific ancestry. When this is the case, and a causal effect is assigned to one of these low-frequency variants, it may be assumed that this variant absent in a given ancestry is uncorrelated with other variants for the same ancestry. Therefore, the r_{i,j} correlation factors for the correlation between the low-frequency variants and all other variants may be set to zero.

Once the one or more iterations have been completed, the method comprises a step S14 of, for each genetic variant, determining the prediction effect size 14 of the genetic variant on one or more of the phenotypes or phenotype combinations based on an average across at least a subset of the iterations of the sampled effect sizes 12 of the genetic variant on the one or more phenotypes or phenotype combinations. The prediction effect size 14 may also be based on an average of posterior effect sizes of the genetic variant for the input unit calculated using the sampled effect sizes 12. The average in either case is taken across at least a subset of the iterations. Any suitable method for averaging may be used. Using multiple iterations and averaging the results overcomes the randomness of the effect size sampling. Once the set of causal variants and their prediction effect sizes 14 has been determined, it becomes straightforward to determine a PRS based on the prediction effect sizes 14. In an embodiment, the average of the sampled effect sizes may be a weighted average, where the sampled effect size of each variant determined to be causal is weighted by a posterior probability that the variant is causal.

For example, the average effect size βᵢ for variant i may be calculated as:

$$\\ \begin{matrix}\underline{{β}_{i}} = \frac{1}{L} {\sum }_{l = 1}^{L} {p}_{i , l} {β}_{i , l} & ( 8 )\end{matrix}$$

where L denotes the total number of iterations, optionally after some initial burn in iterations. The posterior probability that the variant is causal can be determined in any suitable way. For example, it may be determined using the number of iterations in which the variant was determined to be causal, as a proportion of the total number of iterations carried out. Alternatively, the posterior probability that the variant is causal may be calculated from, at every iteration, the probability of the information from the plurality of input units assuming that the variant is causal, and the probability of the information from the plurality of input units assuming that the variant is not causal, as shown in the Bayes factor computation (4).

Typically, the method performs best if the variation in the size of the groups of individuals from which the input units 10 are determined is not too large. For example, when two input units 10 are used derived from a smaller and a larger group of individuals, a significant performance improvement is generally observed once that the smaller group of individuals is about ˜20% or greater of the size of the larger group of individuals.

In some embodiments, one or more of the sampled effect sizes 12 for each genetic variant may be discarded and not included in the average used to obtain the prediction effect sizes 14, i.e. the sampled effect sizes from only a subset of the iterations are used. The number not included may be predetermined, or based on the value of the sampled effect size 12. The discarded sampled effect sizes 12 may be those from the first iterations of the method, for example the first ten iterations, the first twenty iterations, or some other predetermined number. These are often referred to as “burn-in” iterations, and are usually discarded because sampling techniques such as a Monte-Carlo Gibbs sampler take several iterations to converge to a useful sampling pattern.

Given the desirability of determining PRS in general, the present invention can also be used in a method of determining a polygenic risk score for a target phenotype or target phenotype combination for a target individual, as illustrated in FIG. 3. The improved estimates of effect sizes obtained using the methods described above allow for the determination of more accurate PRSs.

The method of determining a PRS comprises a step S20 of receiving genetic information 16 about a region of interest of the genome of the target individual. This may comprise information about the genetic variants (such as single-nucleotide polymorphisms, deletions of insertions) expressed by the individual in the region of interest.

The method further comprises a step S22 of receiving prediction effect sizes 14 on the target phenotype or target phenotype combination of a plurality of genetic variants in the region of interest determined using the method of analysing genetic data described above.

The method further comprises a step S24 of determining the polygenic risk score 20 based on the genetic information for the target individual 16 and the prediction effect sizes 14.

In an embodiment, the PRS 20 is calculated as follows:

$$\\ \begin{matrix}P R S = {\sum }_{k = 1}^{K} {α}_{k} {x}_{k} & ( 9 )\end{matrix}$$

where K is the number of variants that contribute to the PRS 20, xₖ is the genotype for variant k, and αₖ is the PRS weight for variant k, which quantifies the predictive impact of variant k on the target phenotype or phenotype combination (i.e. quantifying the strength of association of variant k on the target phenotype or phenotype combination). Typically the PRS weight αₖ is simply the average effect size for variant k as calculated above, i.e. βₖ.

The method of analysing genetic data may be carried out by an apparatus for analysing genetic data about an organism, also illustrated in FIG. 1. The apparatus comprises a receiving unit 200 configured to receive a plurality of input units 10, each input unit comprising information about the association between a plurality of genetic variants in a region of interest of the genome of the organism and one of a plurality of phenotypes or phenotype combinations of the organism. The apparatus further comprises a data processing unit 210 configured to carry out one or more iterations comprising, for each of the plurality of genetic variants, determining for which of the plurality of phenotypes or phenotype combinations the genetic variant is causal based on the plurality of input units 10, and if the genetic variant is determined to be causal for one or more of the phenotypes or phenotype combinations, determining a sampled effect size 12 of the genetic variant on the one or more phenotypes or phenotype combinations based on the plurality of input units 10 and information about correlations between the plurality of genetic variants in the region of interest. The data processing unit 210 is further configured to, for each genetic variant, determine a prediction effect size 14 of the genetic variant on one or more of the phenotypes or phenotype combinations based on an average across at least a subset of the iterations of the sampled effect sizes 12 of the genetic variant on the one or more phenotypes or phenotype combinations or of posterior effect sizes of the genetic variant for the input unit calculated using the sampled effect sizes.

The invention may also be embodied in a computer program comprising instructions which, when the program is executed by a computer, cause the computer to carry out the method of analysing genetic data. The invention may also be embodied in a computer-readable medium comprising instructions which, when executed by a computer, cause the computer to carry out the method of analysing genetic data.

### Results

As an illustrative example, the present method was applied to predict ischemic stroke in the UK Biobank cohort.

We meta analysed GWAS studies on ischemic stroke from the MEGASTROKE consortium (34,217 cases and 406,111 controls), FinnGen consortium (6,462 cases and 125,569 controls), UK Biobank (3,216 cases and 168,269 controls), and Biobank Japan (17,671 cases and 192,383 controls). Considering this meta-analysis of a single trait in isolation and applying an existing method, the predictive accuracy (quantified using the area under the curve (AUC)) was 0.576 (95% CI 0.565 to 0.587) in individuals of European ancestry.

The present method was applied to combine the ischemic stroke meta-analysis with a separate hypertension meta-analysis [GERA (31,000 cases and 30847 controls) and UKBB (61,925 cases and 108,249 controls)]. This combined analysis resulted in an improved AUC of 0.599 (95% CI 0.589 to 0.610) in individuals of European ancestry in the testing set, demonstrating the advantage of the present method.

### REFERENCES

Bayesian meta-analysis across genome-wide association studies of diverse phenotypes, Trochet H, Pirinen M, Band G, Jostins L, McVean G, Spencer C, Genetic Epidemiology 2019
Multi-trait analysis of genome-wide association summary statistics using MTAG, P Turley et al. Nature Genetics 2018
Vilhjálmsson B J, Yang J, Finucane H K, et al. Modeling Linkage Disequilibrium Increases Accuracy of Polygenic Risk Scores. Am J Hum Genet 2015.
Variable prediction accuracy of polygenic scores within an ancestry group, Hakhamanesh Mostafavi, Arbel Harpak Ipsita Agarwal, Dalton Conley, Jonathan K Pritchard, Molly Przeworski, eLife, 2020
Bycroft et al, The UK Biobank resource with deep phenotyping and genomic data, Nature 2018
A correction for sample overlap in genome-wide association studies in a polygenic pleiotropy-informed framework, Marissa LeBlanc, Verena Zuber, Wesley K. Thompson, Ole A. Andreassen, Schizophrenia and Bipolar Disorder Working Groups of the Psychiatric Genomics Consortium, Arnoldo Frigessi, and Bettina Kulle Andreassen, 2018
Multitrait analysis of glaucoma identifies new risk loci and enables polygenic prediction of disease susceptibility and progression, Jamie E. Craig et al, Nature Genetics 2020

1. A computer-implemented method of analysing genetic data about an organism, the method comprising:
receiving a plurality of input units, wherein each input unit comprises information about the association between a plurality of genetic variants in a region of interest of the genome of the organism and one of a plurality of phenotypes or phenotype combinations of the organism;
carrying out one or more iterations comprising, for each of the plurality of genetic variants:
determining for which of the plurality of phenotypes or phenotype combinations the genetic variant is causal based on the plurality of input units; and
if the genetic variant is determined to be causal for one or more of the phenotypes or phenotype combinations, determining a sampled effect size of the genetic variant on each of the one or more phenotypes or phenotype combinations based on the plurality of input units and information about correlations between the plurality of genetic variants in the region of interest; and
for each genetic variant, determining a prediction effect size of the genetic variant on one or more of the phenotypes or phenotype combinations based on an average across at least a subset of the iterations of the sampled effect sizes of the genetic variant on the one or more phenotypes or phenotype combinations or of posterior effect sizes of the genetic variant for the input unit calculated using the sampled effect sizes.

2. The method of claim 1, wherein determining for which of the plurality of phenotypes or phenotype combinations the genetic variant is causal comprises calculating a plurality of probabilities comprising:
a probability of the information from the plurality of input units assuming that the genetic variant is not causal for any of the phenotypes or phenotype combinations;
a probability of the information from the plurality of input units assuming that the genetic variant is causal for all of the phenotypes or phenotype combinations; and
for one or more subsets of the phenotypes or phenotype combinations, a probability of the information from the plurality of input units assuming that the genetic variant is causal for the subset of phenotypes or phenotype combinations, and
stochastically determining for which of the plurality of phenotypes or phenotype combinations the genetic variant is causal with a probability based on the plurality of probabilities.

3. The method of claim 2, wherein the probability of the information from the plurality of input units assuming that the genetic variant is causal for one or more of the phenotypes or phenotype combinations is dependent on:
a proportion of the plurality of genetic variants expected to be causal;
the plurality of input units; and
a correlation between the effect sizes of the genetic variant on the phenotypes or phenotype combinations.

4. The method of claim 2, wherein the probability of the information from the plurality of input units assuming that the genetic variant is not causal for any of the phenotypes or phenotype combinations is dependent on:
a proportion of the plurality of genetic variants expected to be causal; and
the plurality of input units.

5. The method of claim 2, wherein, for each of the one or more subsets of the phenotypes or phenotype combinations, the probability of the information from the plurality of input units assuming that the genetic variant is causal for the subset of phenotypes or phenotype combinations is dependent on:
a proportion of the plurality of genetic variants expected to be causal;
a subset of input units comprising the input units comprising information about the association between the plurality of genetic variants and one of the subset of phenotypes or phenotype combinations; and
a correlation between the effect sizes of the genetic variant on the phenotypes or phenotype combinations.

6. The method of claim 3, wherein the proportion of the plurality of genetic variants expected to be causal is predetermined.

7. The method of claim 3, wherein the correlation between the effect sizes of the genetic variant on the phenotypes or phenotype combinations is predetermined.

8. The method of claim 3, wherein the proportion of the plurality of genetic variants expected to be causal is updated at each iteration.

9. The method of claim 3, wherein the correlation between the effect sizes of the genetic variant on the phenotypes is updated at each iteration.

10. The method of claim 2, wherein the input units are determined from respective groups of individuals, and each of the plurality of probabilities is dependent on one or more parameters quantifying an overlap in the groups of individuals between respective pairs of input units.

11. The method of claim 1, wherein determining the sampled effect size of the genetic variant comprises calculating a probability distribution, for example a multivariate normal distribution, of effect sizes of the genetic variant on the one or more phenotypes or phenotype combinations, and sampling values of the effect sizes from the probability distribution.

12. (canceled)

13. The method of claim 11, wherein the sampling of values of the effect size is performed using a Monte-Carlo Gibbs sampler.

14. The method of claim 11, wherein the sampling of values of the effect size in each iteration is dependent on the sampled effect sizes from one or more previous iterations.

15. The method of claim 11, wherein the probability distribution is dependent on a correlation between the effect sizes of the genetic variant on the phenotype or phenotype combinations.

16. The method of claim 15, wherein the correlation between the effect sizes of the genetic variant on the phenotypes or phenotype combinations is either predetermined or updated at each iteration.

17. (canceled)

18. The method of claim 1, wherein determining the sampled effect sizes comprises using a model of causal relationships between the plurality of phenotypes or phenotype combinations.

19. The method of claim 1, wherein:
each of the one or more iterations further comprises, for each genetic variant determined to be causal, subtracting weighted effect sizes from the information about the association between each other genetic variant and the phenotype or phenotype combination of each input unit;
the weighted effect sizes being the sampled effect size of the genetic variant on the phenotype or phenotype combination of the input unit weighted by respective correlation factors between the genetic variant and each other genetic variant; and
the correlation factors are determined based on the information about correlations between the plurality of genetic variants in the region of interest.

20. The method of claim 1, wherein carrying out one or more iterations comprises carrying out a predetermined number of iterations.

21. The method of claim 1, wherein each of the one or more iterations further comprises a step of evaluating a convergence parameter, and carrying out one or more iterations comprises carrying out iterations until a predetermined condition on the convergence parameter is met.

22. The method of claim 1, wherein the information about the association between the plurality of genetic variants and each of the phenotypes or phenotype combinations comprises, for each of the plurality of genetic variants, an estimate of a strength of association between the genetic variant and the phenotype or phenotype combination and an error in the estimate of the strength of association.

23. A method of determining a polygenic risk score for a target phenotype or target phenotype combination for a target individual comprising:
receiving genetic information about a region of interest of the genome of the target individual;
receiving prediction effect sizes on the target phenotype or target phenotype combination of a plurality of genetic variants in the region of interest determined using the method of analysing genetic data of claim 1; and
determining the polygenic risk score based on the genetic information for the target individual and the prediction effect sizes.

24. An apparatus for analysing genetic data about an organism, the apparatus comprising:
a receiving unit configured to receive a plurality of input units, wherein each input unit comprises information about the association between a plurality of genetic variants in a region of interest of the genome of the organism and one of a plurality of phenotypes or phenotype combinations of the organism; and
a data processing unit configured to:
carry out one or more iterations comprising, for each of the plurality of genetic variants:
determining for which of the plurality of phenotypes or phenotype combinations the genetic variant is causal based on the plurality of input units; and
if the genetic variant is determined to be causal for one or more of the phenotypes or phenotype combinations, determining a sampled effect size of the genetic variant on the one or more phenotypes or phenotype combinations based on the plurality of input units and information about correlations between the plurality of genetic variants in the region of interest; and
for each genetic variant, determining a prediction effect size of the genetic variant on one or more of the phenotypes or phenotype combinations based on an average across at least a subset of the iterations of the sampled effect sizes of the genetic variant on the one or more phenotypes or phenotype combinations or of posterior effect sizes of the genetic variant for the input unit calculated using the sampled effect sizes.

25. A computer program or a computer-readable medium comprising instructions which, when executed by a computer, causes the computer to carry out the method of claim 1.

26. (canceled)
